(19) United States
(12) Patent Application Publication
(10) Pub. No.: US 2025/0241832 A1
(43) Pub. Date: Jul. 31, 2025

(54) MAKEUP COSMETIC COMPOSITION FOR ABSORBING SEBUM AND METHOD FOR PREPARING SAME

(71) Applicant: Amorepacific Corporation, Seoul (KR)

(72) Inventors: Sanghoon KIM, Yongin-si (KR); Taewon SEO, Yongin-si (KR); Seung Hwan YI, Yongin-si (KR)

(73) Assignee: Amorepacific Corporation, Seoul (KR)

(21) Appl. No.: 18/855,669

(22) PCT Filed: Mar. 22, 2023
(86) PCT No.: PCT/KR2023/003784

§ 371 Date: Oct. 10, 2024

(30) Foreign Application Priority Data
Apr. 19, 2022 (KR) 10-2022-0048500
Oct. 17, 2022 (KR) 10-2022-0133056

(51) Int. Cl. A61K 8/02; A61Q 19/00

(52) U.S. Cl. CPC A61K 8/0279; A61Q 19/008

(57) ABSTRACT

Disclosed are: a cosmetic composition for absorbing sebum, comprising a wax-coated porous powder comprising a porous powder and a wax coating layer formed on the surface thereof, wherein the surface of the porous powder is an outer surface and an inner surface; and a method for preparing the cosmetic composition, the method comprising the steps of: (a) forming a wax coating layer on the outer surface and the inner surface of the porous powder; (b) supporting a volatile oil inside the powder, and (c) dispersing a result product of step (a) in a non-volatile solvent.

### CROSS REFERENCE TO RELATED APPLICATION

The present application claims the priorities of Korean Patent Application No. 10-2022-0048500 filed on Apr. 19, 2022, and Korean Patent Application No. 10-2022-0133056 filed on Oct. 17, 2022, the entire contents of which is incorporated herein for all purposes by this reference.

The present disclosure relates to a cosmetic composition for absorbing sebum, comprising a porous powder having a wax-coated film formed thereon and a method for preparing the same.

### BACKGROUND ART

The persistency that allows makeup to maintain a makeup state similar to the original for a long time is a very important attribute in the makeup. To this end, various techniques are used, such as using a film former, applying surface modification to increase the skin adhesion of a pigment, and using an absorbent powder.

The absorbent powder mainly includes a powder with a porous or hollow structure, and is used for the purpose of absorbing sebum secreted from the skin into pores of the powder to prevent sebum from deforming the makeup film. The higher a content of this absorbent powder, the less a degree to which the makeup film is damaged by sebum, but at the same time, there is a limit to increasing the content indefinitely due to strong disadvantages such as stickiness and roughness when being applied to the skin. Therefore, in case the same amount of the absorbent powder is used, depending on how many pores and large surface area the powder has, the effect of increasing absorbability is achieved even with a small content thereof.

The most commonly used absorbent powder is a silica or a material modified based on the silica, which has high absorbability. However, since many absorbent powders containing the silica or silicone series generally have poor affinity between their surface and sebum, there is a limitation that the actual absorption amount does not have the best effect compared to the large surface area of the pores.

### DISCLOSURE

### Technical Problem

In an aspect, the present disclosure is intended to provide a pre-dispersed composition for preparing a cosmetic composition for absorbing sebum.

In an aspect, the present disclosure is intended to provide a cosmetic composition for absorbing sebum.

In an aspect, the present disclosure is intended to provide a cosmetic composition comprising a porous powder for absorbing sebum.

In an aspect, the present disclosure is intended to provide a method for preparing a cosmetic composition for absorbing sebum.

### Technical Solution

In order to achieve the above purpose, the following aspects are provided:

In an aspect, the present invention provides a pre-dispersed composition for preparing a cosmetic composition for absorbing sebum, the pre-dispersed composition comprising a porous powder; a wax; and a volatile oil, wherein the porous powder and the wax are dispersed in the volatile oil.

In an aspect, the present invention provides a cosmetic composition for absorbing sebum, which comprises a wax-coated porous powder containing a porous powder and a wax coating layer formed on a surface thereof, wherein the surface of the porous powder is an outer surface and an inner surface.

In an aspect, the present invention provides a method for preparing the cosmetic composition, comprising the steps of: (a) preparing a pre-dispersed composition; and (b) dispersing a result product of step (a) in a non-volatile solvent.

### Advantage Effects

According to an aspect of the present invention, a cosmetic composition for absorbing sebum, which comprises a porous powder having a wax-coated film formed thereon, better absorbs sebum by forming the coating film on an inner surface of an absorbent powder that captures sebum, thereby increasing the persistency, which is an important attribute of makeup, even if a small amount of the absorbent powder is contained.

### DESCRIPTION OF THE DRAWINGS

FIG. 1 is a schematic diagram of cases (a to c) where a wax-coated film is not properly formed on a porous hollow powder and cases (d and e) where the wax-coated film is formed.

FIG. 2 is a schematic diagram of sebum adsorption for cases (a to c) when a wax-coated film is not properly formed on a porous hollow powder and cases (d and e) where the wax-coated film is formed.

FIG. 3 is an image of an absorption amount of sebum observed after being applied to a skin model.

### MODE FOR INVENTION

The term “porous” used in this specification refers to a property in which many small holes (pores) exist on a surface or inside of the powder, and includes a hollow type in which the inside of the powder is empty.

The term “outer surface” used in this specification refers to a surface outside the powder where one powder particle comes into contact with an external air. Also, the term “inner surface” refers to a surface inside the porous powder particle excluding the outer surface of the powder, that is, a surface of the internal pores, and is used to include a surface of the internal channels formed by connecting the pores.

The term “non-silicone-based wax” used in this specification refers to all types of the wax that do not include silicones such as trimethicone, dimethicone, and silane in their chemical names, that is, do not contain silicone components, and may include, for example, polyethylene, tribehenin, microcrystalline wax, ozokerite, etc.

The term “average thickness” of the wax coating layer used in this specification refers to a thickness obtained by measuring a thickness of the wax coating layer formed at five arbitrary points on the outer surface of the porous powder and calculating their average.

Hereinafter, the present invention will be described in detail.

In an aspect, the present invention provides a pre-dispersed composition for preparing a cosmetic composition for absorbing sebum, the pre-dispersed composition comprising a porous powder: a wax; and a volatile oil, wherein the porous powder and the wax are dispersed in the volatile oil.

In an embodiment, a sum of the contents of the porous powder and the wax in the pre-dispersed composition may be 2 to 20 wt % based on the total weight of the pre-dispersed composition. For example, the contents may be 2 wt % or more, 2.5 wt % or more, 3 wt % or more, 3.5 wt % or more, 4 wt % or more, 4.5 wt % or more, 5 wt % or more, 5.5 wt % or more, 6 wt % or more, 6.5 wt % or more, 7 wt % or more, 7.5 wt % or more, 8 wt % or more, 8.5 wt % or more, 9 wt % or more, 9.5 wt % or more, 10 wt % or more, and may be 20 wt % or less, 19.5 wt % or less, 19 wt % or less, 18.5 wt % or less, 18 wt % or less, 17.5 wt % or less, 17 wt % or less, 16.5 wt % or less, 16 wt % or less, 15.5 wt % or less, 15 wt % or less, 14.5 wt % or less, 14 wt % or less, 13.5 wt % or less, 13 wt % or less, 12.5 wt % or less, 12 wt % or less, 11.5 wt % or less, 11 wt % or less, or 10.5 wt % or less.

In an embodiment, a weight ratio of the porous powder:the wax:the volatile oil in the pre-dispersed composition may be 1:0.25-6:13-23. For example, the weight ratio may be 1:0.25 or more, 0.3 or more, 0.35 or more, 0.4 or more, 0.45 or more, 0.5 or more, 0.55 or more, 0.6 or more, 0.65 or more, 0.7 or more, 0.75 or more, 0.8 or more, 0.85 or more, 0.9 or more, 0.95 or more, or 1 or more, and 6 or less, 5.5 or less, 5 or less, 4.5 or less, 4 or less, 3.75 or less, 3.5 or less, 3.25 or less, 3 or less, 2.75 or less, 2.5 or less, 2.25 or less, 2 or less, 1.75 or less, 1.5 or less, or 1.25 or less: 13 or more, 14 or more, 15 or more, 16 or more, 17 or more, or 18 or more, and 23 or less, 22 or less, 21 or less, 20 or less, or 19 or less. If the volatile oil is too small, fluidity of the mixture is low to make it difficult to perform effective and uniform coating. On the other hand, if the volatile oil is too large, there is less opportunity for the porous powder and the wax to meet each other, thereby making it difficult to perform proper coating.

In an embodiment, the porous powder may be dispersed separately from the wax in the pre-dispersed composition, or may be coated on a surface of the porous powder with the wax. For example, the porous powder may not initially contain a wax coating layer, and may gradually be coated on the surface of the porous powder with the wax.

In an embodiment, the porous powder may be a wax-coated porous powder having the wax coating layer on its surface, wherein the surface is an outer surface and an inner surface.

In an embodiment, the wax coating layer may be formed on at least one of the outer surface and the inner surface of the porous powder. Specifically, the wax coating layer may be uniformly formed on a surface of the pores formed by the porous and/or hollow structure on the outer surface of the porous powder as well as the inner surface of the porous powder.

In an embodiment, an average thickness of the wax coating layer may be 0.01% to 20% of a diameter of the porous powder. Specifically, the average thickness of the wax coating layer is obtained by measuring a thickness of the wax coating layer formed at five arbitrary points on the outer surface of the porous powder and calculating their average. For example, the average thickness of the coating layer may be 0.01% or more, 0.02% or more, 0.03% or more, 0.04% or more, 0.05% or more, 0.06% or more, 0.07% or more, 0.08% or more, 0.09% or more, 0.1% or more, 0.15% or more, 0.2% or more, 0.3% or more, 0.4% or more, 0.5% or more, 0.6% or more, 0.7% or more, 0.8% or more, 0.9% or more, 1% or more, 1.5% or more, 2% or more, 2.5% or more, 3% or more, 3.5% or more, 4% or more, 4.5% or more, 5% or more, or 10% or more of the diameter of the porous powder, and may be 20% or less, 19.5% or less, 19% or less, 18.5% or less, 18% or less, 17.5% or less, 17% or less, 16.5% or less, 16% or less, 15.5% or less, 15% or less, 14.5% or less, 14% or less, 13.5% or less, 13% or less, 12.5% or less, 12% or less, 11.5% or less, 11% or less, or 10.5% or less of the diameter of the porous powder.

In an embodiment, a thickness of the wax coating layer may be such that, when the thickness of the coating layer is measured at any point of the porous powder, 95% or more may exhibit the thickness within the above range. That is, the wax is coated on the porous powder with a uniform thickness.

In an embodiment, the pre-dispersed composition may be the one that the volatile oil is supported within the pores of the wax-coated porous powder. Specifically, the volatile oil within the wax-coated porous powder may be filled in an internal space of the pores of the wax-coated porous powder and be evaporated when the cosmetic composition is applied to the skin, wherein sebum is absorbed within the pores of the wax-coated porous powder in which the volatile oil has evaporated.

In an embodiment, the wax-coated porous powder may have an absorption amount of 0.002 to 0.03 ml/g based on the weight of the uncoated porous powder. For example, it may have an absorption amount of 0.002 ml/g or more, 0.0025 ml/g or more, 0.003 ml/g or more, 0.0035 ml/g or more, 0.004 ml/g or more, 0.0045 ml/g or more, 0.005 ml/g or more, 0.0055 ml/g or more, 0.006 ml/g or more, 0.0065 ml/g or more, 0.007 ml/g or more, 0.0075 ml/g or more, 0.008 ml/g or more, 0.0085 ml/g or more, 0.009 ml/g or more, 0.0095 ml/g or more, or 0.01 ml/g or more, and may have an absorption amount of 0.03 ml/g or less, 0.029 ml/g or less, 0.028 ml/g or less, 0.027 ml/g or less, 0.026 ml/g or less, 0.025 ml/g or less, 0.024 ml/g or less, 0.023 ml/g or less, 0.022 ml/g or less, 0.021 ml/g or less, 0.02 ml/g or less, 0.019 ml/g or less, 0.018 ml/g or less, 0.017 ml/g or less, 0.016 ml/g or less, 0.015 ml/g or less, 0.014 ml/g or less, 0.013 ml/g or less, 0.012 ml/g or less, or 0.011 ml/g or less.

In an embodiment, the porous powder may have a surface area of 10 to 500 m²/g. For example, the surface area of the porous powder may be 10 m²/g or more, 15 m²/g or more, 20 m²/g or more, 25 m²/g or more, 30 m²/g or more, 35 m²/g or more, 40 m²/g or more, 45 m²/g or more, 50 m²/g or more, 55 m²/g or more, 60 m²/g or more, 65 m²/g or more, 70 m²/g or more, 75 m²/g or more, 80 m²/g or more, 85 m²/g or more, 90 m²/g or more, 95 m²/g or more, 100 m²/g or more, or 110 m²/g or more, and may be 500 m²/g or less, 450 m²/g or less, 400 m²/g or less, 350 m²/g or less, 300 m²/g or less, 250 m²/g or less, 200 m²/g or less, 190 m²/g or less, 180 m²/g or less, 170 m²/g or less, 160 m²/g or less, 150 m²/g or less, 140 m²/g or less, 130 m²/g or less, or 120 m²/g or less.

In an embodiment, the wax may be a non-silicone-based wax. The non-silicone-based wax has a high affinity for sebum and thus can improve the absorption capacity of the porous powder. The non-silicone-based wax refers to a wax that does not include silicones such as trimethicone, dimethicone, and silane in its chemical name. For example, it may include, but is not limited to, polyethylene, tribehenin, microcrystalline wax, ozokerite, and a combination thereof.

In an embodiment, the wax may have a melting point of 40 to 150° C. For example, the wax may have a melting point of 40° C. or higher, 45° C. or higher, 50° C. or higher, 55° C. or higher, 60° C. or higher, 65° C. or higher, 70° C. or higher, 75° C. or higher, 80° C. or higher, 85° C. or higher, 90° C. or higher, 95° C. or higher, or 100° C. or higher, and a melting point of 150° C. or lower, 145° C. or lower, 140° C. or lower, 135° C. or lower, 130° C. or lower, 125° C. or lower, 120° C. or lower, 115° C. or lower, or 110° C. or lower.

In an embodiment, the wax may have excellent affinity for sebum without any separation after 24 hours from the point of time when a sebum oil and the wax are mixed in a weight ratio of 7:3 under a molten state at 90° C. and then reach a room temperature by natural cooling. The sebum oil may be sebum secreted from the human body or an oil similar to it, wherein the oil similar to sebum may include, for example, caprylic/capric triglyceride, etc.

In an embodiment, the volatile oil may have a temperature vapor pressure of 0.3 to 90 mmHg at 40° C. For example, the volatile oil may have a temperature vapor pressure of 0.3 mmHg or more, 0.5 mmHg or more, 0.8 mmHg or more, 1 mmHg or more, 2 mmHg or more, 3 mmHg or more, 4 mmHg or more, 5 mmHg or more, 6 mmHg or more, 7 mmHg or more, 8 mmHg or more, 9 mmHg or more, 10 mmHg or more, 15 mmHg or more, 20 mmHg or more, 25 mmHg or more, 30 mmHg or more, 35 mmHg or more, 40 mmHg or more, or 45 mmHg or more, and may have a temperature vapor pressure of 90 mmHg or less, 85 mmHg or less, 80 mmHg or less, 75 mmHg or less, 70 mmHg or less, 65 mmHg or less, 60 mmHg or less, 55 mmHg or less, or 50 mmHg or less. However, a low volatile oil that exceeds the above range of the temperature vapor pressure may also be used at 50 wt % or less based on the total weight of the oil for the purpose of adjusting the temperature vapor pressure of a high volatile oil. The volatile oil that may be used include, but are not limited to, cyclopentasiloxane, cyclohexasiloxane, etc.

In an embodiment, the pre-dispersed composition may further comprise a fragrance, a pigment, a dispersant, a stabilizer, a pH regulator, a preservative, etc., within a range that does not impair the purpose of the present invention.

In an embodiment, the cosmetic composition may be a makeup cosmetic composition.

In an aspect, the present invention provides a cosmetic composition for absorbing sebum, which comprises a wax-coated porous powder containing a porous powder and a wax coating layer formed on a surface thereof, wherein the surface of the porous powder is an outer surface and an inner surface.

In an embodiment, the wax coating layer may be formed on at least one of the outer surface and the inner surface of the porous powder. Specifically, the wax coating layer may be uniformly formed on the outer surface of the porous powder as well as on the surface of pores formed by porous and/or hollow structures inside the porous powder.

In an embodiment, a weight ratio of the wax and the porous powder may be 1:0.25-6. For example, the above weight ratio may be 1:0.25 or more, 1:0.3 or more, 1:0.35 or more, 1:0.4 or more, 1:0.45 or more, 1:0.5 or more, 1:0.55 or more, 1:0.6 or more, 1:0.65 or more, 1:0.7 or more, 1:0.75 or more, 1:0.8 or more, 1:0.85 or more, 1:0.9 or more, 1:0.95 or more, or 1:1 or more, and may be 1:6 or less, 1:5.5 or less, 1:5 or less, 1:4.5 or less, 1:4 or less, 1:3.5 or less, 1:3 or less, 1:2.5 or less, 1:2 or less, 1:1.5 or less, 1:1.4 or less, 1:1.3 or less, 1:1.2 or less, or 1:1.1 or less. If the weight ratio of the wax to the porous powder is less than 1:0.25, the wax may be excessively coated on the surface of the porous powder in an internal space of the powder to reduce a space capable of absorbing sebum. In addition, if the weight ratio exceeds 1:4, the wax may not be properly coated on the porous powder to reduce the absorption amount by preventing sebum from entering pores of the powder due to the property of the porous powder itself that has low affinity for sebum.

In an embodiment, the wax may be a non-silicone-based wax. The non-silicone-based wax has a high affinity for sebum and thus can improve the absorption capacity of the porous powder. The non-silicone-based wax refers to a wax that does not include silicones such as trimethicone, dimethicone, and silane in its chemical name. For example, it may include, but is not limited to, polyethylene, tribehenin, microcrystalline wax, ozokerite, and a combination thereof.

In an embodiment, the wax may have a melting point of 40 to 150° C. For example, the wax may have a melting point of 40° C. or higher, 45° C. or higher, 50° C. or higher, 55° C. or higher, 60° C. or higher, 65° C. or higher, 70° C. or higher, 75° C. or higher, 80° C. or higher, 85° C. or higher, 90° C. or higher, 95° C. or higher, or 100° C. or higher, and a melting point of 150° C. or lower, 145° C. or lower, 140° C. or lower, 135° C. or lower, 130° C. or lower, 125° C. or lower, 120° C. or lower, 115° C. or lower, or 110° C. or lower.

In an embodiment, the wax may have excellent affinity for sebum without any separation after 24 hours from the point of time when a sebum oil and the wax are mixed in a weight ratio of 7:3 under a molten state at 90° C. and then reach a room temperature by natural cooling. The sebum oil may be sebum secreted from the human body or an oil similar to it, and may include, for example, caprylic/capric triglyceride, etc.

In an embodiment, the porous powder may be silica series. For example, it may include silica, silica silylate, silica dimethicone silylate, and a combination thereof, but is not limited thereto.

In an embodiment, the porous powder may have a surface area of 10 to 500 m²/g. For example, the surface area of the porous powder may be 10 m²/g or more, 15 m²/g or more, 20 m²/g or more, 25 m²/g or more, 30 m²/g or more, 35 m²/g or more, 40 m²/g or more, 45 m²/g or more, 50 m²/g or more, 55 m²/g or more, 60 m²/g or more, 65 m²/g or more, 70 m²/g or more, 75 m²/g or more, 80 m²/g or more, 85 m²/g or more, 90 m²/g or more, 95 m²/g or more, 100 m²/g or more, or 110 m²/g or more, and may be 500 m²/g or less, 450 m²/g or less, 400 m²/g or less, 350 m²/g or less, 300 m²/g or less, 250 m²/g or less, 200 m²/g or less, 190 m²/g or less, 180 m²/g or less, 170 m²/g or less, 160 m²/g or less, 150 m²/g or less, 140 m²/g or less, 130 m²/g or less, or 120 m²/g or less.

In an embodiment, the wax-coated porous powder may be contained in an amount of more than 0.5 wt % to less than or equal to 5 wt % based on the total weight of the cosmetic composition. For example, it may be contained in an amount of more than 0.5 wt %, 0.55 wt % or more, 0.6 wt % or more, 0.65 wt % or more, 0.7 wt % or more, 0.75 wt % or more, 0.8 wt % or more, 0.85 wt % or more, 0.9 wt % or more, 0.95 wt % or more, or 1 wt % or more, and may be contained in an amount of 5 wt % or less, 4.75 wt % or less, 4.5 wt % or less, 4.25 wt % or less, 4 wt % or less, 3.75 wt % or less, 3.5 wt % or less, 3.25 wt % or less, 3 wt % or less, 2.75 wt % or less, 2.5 wt % or less, 2.25 wt % or less, 2 wt % or less, 1.75 wt % or less, or 1.5 wt % or less.

In an embodiment, a thickness of the wax coating layer may be 0.01% to 20% of a diameter of the porous powder. Specifically, an average thickness of the wax coating layer is obtained by measuring a thickness of the wax coating layer formed at five arbitrary points on the outer surface of the porous powder and calculating their average.

In an embodiment, the wax-coated porous powder may have an absorption amount of 0.002 to 0.03 ml/g based on the weight of the uncoated porous powder. For example, it may have an absorption amount of 0.002 ml/g or more, 0.0025 ml/g or more, 0.003 ml/g or more, 0.0035 ml/g or more, 0.004 ml/g or more, 0.0045 ml/g or more, 0.005 ml/g or more, 0.0055 ml/g or more, 0.006 ml/g or more, 0.0065 ml/g or more, 0.007 ml/g or more, 0.0075 ml/g or more, 0.008 ml/g or more, 0.0085 ml/g or more, 0.009 ml/g or more, 0.0095 ml/g or more, or 0.01 ml/g or more, and may have an absorption amount of 0.03 ml/g or less, 0.029 ml/g or less, 0.028 ml/g or less, 0.027 ml/g or less, 0.026 ml/g or less, 0.025 ml/g or less, 0.024 ml/g or less, 0.023 ml/g or less, 0.022 ml/g or less, 0.021 ml/g or less, 0.02 ml/g or less, 0.019 ml/g or less, 0.018 ml/g or less, 0.017 ml/g or less, 0.016 ml/g or less, 0.015 ml/g or less, 0.014 ml/g or less, 0.013 ml/g or less, 0.012 ml/g or less, or 0.011 ml/g or less.

In an embodiment, the cosmetic composition may further comprise a volatile oil.

In an embodiment, the wax-coated porous powder may have the volatile oil supported within pores thereof. Specifically, the volatile oil within the wax-coated porous powder may be filled in an internal space of the pores of the wax-coated porous powder and be evaporated when the cosmetic composition is applied to the skin, wherein sebum is absorbed within the pores of the wax-coated porous powder in which the volatile oil has been evaporated.

In an embodiment, the volatile oil may have a temperature vapor pressure of 0.3 to 90 mmHg at 40° C. For example, the volatile oil may have a temperature vapor pressure of 0.3 mmHg or more, 0.5 mmHg or more, 0.8 mmHg or more, 1 mmHg or more, 2 mmHg or more, 3 mmHg or more, 4 mmHg or more, 5 mmHg or more, 6 mmHg or more, 7 mmHg or more, 8 mmHg or more, 9 mmHg or more, 10 mmHg or more, 15 mmHg or more, 20 mmHg or more, 25 mmHg or more, 30 mmHg or more, 35 mmHg or more, 40 mmHg or more, or 45 mmHg or more, and may have a temperature vapor pressure of 90 mmHg or less, 85 mmHg or less, 80 mmHg or less, 75 mmHg or less, 70 mmHg or less, 65 mmHg or less, 60 mmHg or less, 55 mmHg or less, or 50 mmHg or less. However, a low volatile oil that exceeds the above range of the temperature vapor pressure may also be used at 50 wt % or less based on the total weight of the oil for the purpose of adjusting the temperature vapor pressure of a high volatile oil. The volatile oil that may be used include, but are not limited to, cyclopentasiloxane, cyclohexasiloxane, etc.

In an embodiment, the cosmetic composition may further comprise a non-volatile solvent. Specifically, the wax-coated porous powder may be dispersed in the non-volatile solvent. For example, the non-volatile solvent may include, but is not limited to, dimethicone, caprylic/capric triglyceride, and the like.

In an embodiment, the cosmetic composition may further comprise a fragrance, a pigment, a dispersant, a stabilizer, a pH regulator, a preservative, etc., within a range that does not impair the purpose of the present invention.

In an embodiment, the cosmetic composition may further comprise a dispersant. Specifically, the dispersant plays a role in helping to uniformly coat an inner surface of the porous powder by increasing fluidity of a composition formulation. In case the volatile oil contained in the composition is a silicone oil, an acrylate/ethylhexylacrylate/dimethicone methacrylate copolymer to which silicone is bound, an acrylate/ethylhexylacrylate/dimethicone methacrylate copolymer, polyethylene glycol, polypropylene glycol, or dimethicone in which a glyceryl group is bound alone or in a combination may be used, but is not limited thereto.

In an embodiment, the cosmetic composition may further comprise a non-absorbent powder. Specifically, the non-absorbent powder is contained to enhance stability of a pre-dispersed composition in which the wax-coated porous powder is pre-dispersed, but a content of the non-absorbent powder may be limited to 0.01 to 80 wt % within a range that does not impair the fluidity in the preparation process by heating. Examples of the non-absorbent powder may include, but are not limited to, barium sulfate, a mica, titanium dioxide, an iron oxide, and the like.

In an embodiment, the cosmetic composition may be a makeup cosmetic composition.

In an embodiment, the makeup cosmetic composition may be a formulation selected from the group consisting of a makeup primer, a foundation, a makeup base, and a tinted moisturizer.

In an embodiment, the cosmetic composition may be prepared from the pre-dispersed composition.

In other aspect, the present invention may include a use of the cosmetic composition for absorbing sebum. Specifically, the composition may be a cosmetic composition for absorbing sebum, comprising a wax-coated porous powder containing a porous powder and a wax coating layer formed on a surface thereof, wherein the surface of the porous powder is an outer surface and an inner surface.

In another aspect of the present invention, a method for absorbing sebum of a subject is provided, which comprises administering an effective amount of the wax-coated porous powder or the cosmetic composition comprising the wax-coated porous powder to the subject in need thereof.

In another aspect of the present invention, a use of a wax-coated porous powder in preparing a cosmetic composition for absorbing sebum is provided.

In another aspect of the present invention, a wax-coated porous powder for absorbing sebum or a cosmetic composition comprising the wax-coated porous powder is provided.

In another aspect, the present invention provides a method for preparing the cosmetic composition, comprising the steps of: (a) preparing a pre-dispersed composition; and (b) dispersing a result product of step (a) in a non-volatile solvent.

In an embodiment, step (a) may include: (i) adding a porous powder and a wax to a volatile oil; and (ii) dispersing the porous powder and the wax using a milling machine or a mixer. Specifically, step (ii) is achieved through high-strength dispersion using a roll-typed process or beads.

In an embodiment, step (b) may include adding the pre-dispersed composition prepared by step (a) in an amount of 2 to 20 wt % based on the total weight of the cosmetic composition. For example, the pre-dispersed composition may be added in an amount of 2 wt % or more, 2.5 wt % or more, 3 wt % or more, 3.5 wt % or more, 4 wt % or more, 4.5 wt % or more, 5 wt % or more, 5.5 wt % or more, 6 wt % or more, 6.5 wt % or more, 7 wt % or more, 7.5 wt % or more, 8 wt % or more, 8.5 wt % or more, 9 wt % or more, 9.5 wt % or more, or 10 wt % or more, and in an amount of 20 wt % or less, 19.5 wt % or less, 19 wt % or less, 18.5 wt % or less, 18 wt % or less, 17.5 wt % or less, 17 wt % or less, 16.5 wt % or less, 16 wt % or less, 15.5 wt % or less, 15 wt % or less, 14.5 wt % or less, 14 wt % or less, 13.5% or less, 13 wt % or less, 12.5 wt % or less, 12 wt % or less, 11.5 wt % or less, 11 wt % or less, or 10.5 wt % or less, based on the total weight of the cosmetic composition.

In an embodiment, step (a) may be performed at 40 to 150° C. Specifically, step (a) is performed at a temperature of the melting point of the wax so as to form a uniform coating layer in a molten state of the wax. However, in order to prevent volatilization of the volatile oil used together during the preparation of the composition, the temperature is lowered within the temperature range capable of melting the wax, and when step (a) is completed, the temperature is lowered again below the melting point of the wax to maintain the coating. For example, step (a) may vary depending on a type of the wax used for coating, but may be performed at a temperature of 40° C. or higher, 45° C. or higher, 50° C. or higher, 55° C. or higher, 60° C. or higher, 65° C. or higher, 70° C. or higher, 75° C. or higher, 80° C. or higher, 85° C. or higher, 90° C. or higher, 95° C. or higher, or 100° C. or higher, and 150° C. or lower, 145° C. or lower, 140° C. or lower, 135° C. or lower, 130° C. or lower, 125° C. or lower, 120° C. or lower, 115° C. or lower, or 110° C. or lower.

Hereinafter, the present invention will be described in more detail through Examples and the like. Since these Examples are intended only to illustrate the present invention, the scope of the present invention is not limited by these Examples. Anything that has substantially the same constitution as the technical idea described in the claims of the present invention and achieves the same operational effect is included in the technical scope of the present invention.

### 1. Preparation of Cosmetic Composition for Absorbing Sebum, Comprising Wax-Coated Porous Powder

A cosmetic composition for absorbing sebum was prepared according to the composition of Table 1 below. Except for the process of preparing a pre-dispersed composition, the cosmetic composition of both of Comparative Examples and Examples were prepared in the same method as the method of preparing a general face makeup product, which comprises first mixing an oil phase containing an emulsifier, dispersing a powder and a pigment to the mixture through homo-mixing, and then emulsifying it by the addition of a dissolved water phase. In case of the pre-dispersed composition of a wax-coated powder, which is the first step of preparing the oil phase, it is produced through high-strength dispersion at the mixing ratio mentioned in the above description. After that, a remaining oil component of the cosmetic composition and other components that disperse in the oil component are added to the composition to further perform the dispersion, whereby the preparation of the oil phase is completed. In case of a wax, it can be separately melted by heating or be naturally melted by heating during the high-strength dispersion process to enable the coating.

TABLE 1

| | Component | Comparative | Comparative | Comparative | Comparative | Example | Example |
|---|---|---|---|---|---|---|---|
| Mixing Purpose | Name | Example 1 | Example 2 | Example 3 | Example 4 | 1 | 2 |
| wax-coated | Cyclopentasiloxane | 9 | 9.5 | — | 9.49 | 9 | 7 |
| porous powder | Tribehenin | — | | | 0.01 | 0.5 | 2.5 |
| (pre—dispersed) | Cetyl | 0.5 | — | — | | — | — |
| | dimethicone | | | | | | |
| | Silica | 0.5 | 0.5 | — | 0.5 | 0.5 | 0.5 |
| Oil | Cyclopentasiloxane | To 100 | To 100 | To 100 | To 100 | To 100 | To 100 |
| Pigment | Titanium | 7 | 7 | 7 | 7 | 7 | 7 |
| | dioxide | | | | | | |
| | Yellow iron | 1 | 1 | 1 | 1 | 1 | 1 |
| | oxide | | | | | | |
| | Red iron oxide | 0.3 | 0.3 | 0.3 | 0.3 | 0.3 | 0.3 |
| | Black iron oxide | 0.1 | 0.1 | 0.1 | 0.1 | 0.1 | 0.1 |
| Film former | Trimethylsiloxy | 3 | 3 | 3 | 3 | 3 | 3 |
| | silicate | | | | | | |
| Comparative | Silica | — | — | 0.5 | — | — | — |
| Example - | Wax | 2.49 | 2.5 | 2.5 | 2.49 | 2 | 0 |
| Example | | | | | | | |
| Fixed total | | | | | | | |
| amount of silica | | | | | | | |
| and wax in | | | | | | | |
| formulation | | | | | | | |
| Emulsifier | PEG—10 | 4 | 4 | 4 | 4 | 4 | 4 |
| | DIMETHICONE | | | | | | |
| Thickener | Disteardiammonium | 1 | 1 | 1 | 1 | 1 | 1 |
| | hectorite | | | | | | |
| Solvent | Purified water | 45 | 45 | 45 | 45 | 45 | 45 |
| Emulsion | NaCl | 1 | 1 | 1 | 1 | 1 | 1 |
| stabilizer | | | | | | | |

| | | | | | | |
|---|---|---|---|---|---|---|
| Total content (%) | 100 | 100 | 100 | 100 | 100 | 100 |

### 2. Sebum Affinity by Each of Wax Type

After a sebum-like oil (caprylic/capric triglyceride) and a wax were mixed in a weight ratio of 7:3 in a molten state at 90° C. stability of the mixed solution was confirmed after 24 hours from the period of time it reached a room temperature by natural cooling.

TABLE 2

| | | Stability (whether or |
|---|---|---|
| Wax Type | | not it is separated) |
| Silicon series | Cetyl dimethicone | Surface crystal |
| | | precipitation |
| | Bis-PEG-18 methyl ether | Oil separation and |
| | dimethyl silane | sedimentation |
| Non-silicon series | Polyethylene | None |
| | Tribehenin | None |
| | Microcrystalline wax | None |
| | Ozokerite | None |

As a result, it was confirmed that the waxes containing silicone such as dimethicone and silane all had poor affinity for sebum because they were precipitated out or separated out, whereas the waxes not containing silicone such as polyethylene, tribehenin, microcrystalline wax, and ozokerite had good affinity for sebum through a stable mixing pattern without separation.

### 3. Confirmation of Absorption Amount of Sebum According to Coated Pattern

### (1) Coated Pattern According to the Added Step and Weight Ratio of a Wax and a Porous Powder

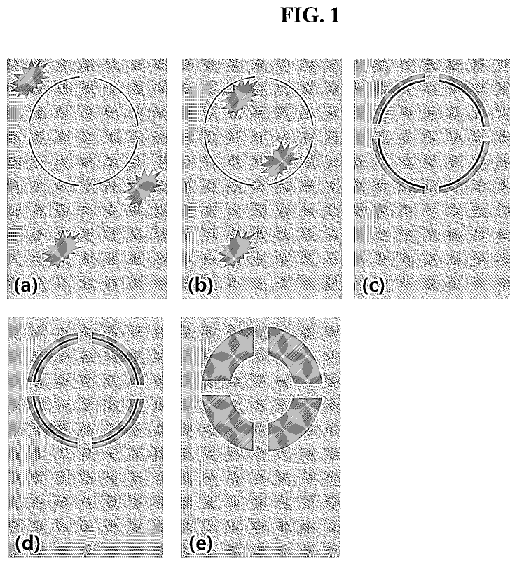

In case a wax was not added together in the pre-dispersion step where a volatile oil and a porous powder were added and dispersed, but was added in the step where it was dispersed in a non-volatile solvent later (Comparative Example 2), a wax coating layer was not formed on the porous powder (silica) as shown in (a) of FIG. 1. In addition, in case the volatile solvent, the wax, and the porous powder were dispersed together in the non-volatile solvent without the pre-dispersion step (Comparative Example 3), the wax coating layer was also not formed on the porous powder as shown in (b) of FIG. 1.

On the other hand, in case the wax, the porous powder, and the volatile oil were added together in the pre-dispersion step (Comparative Example 4, Example 1, and Example 2), the coating layer was formed on the surface of the porous powder as shown in (d)-(e) of FIG. 1.

Meanwhile, in case of Comparative Example 4 where a weight ratio of the wax and the porous powder in the pre-dispersion step was 1:50, the wax was not uniformly coated on an inner surface of the porous powder as shown in (c) of FIG. 1. On the other hand, in case of Examples 1 and 2 where weight ratios of the wax and the porous powder were 1:1 and 1:0.2, respectively, the wax coating layer was uniformly formed on the inner surface of the porous powder as shown in (d)-(e) of FIG. 1.

Further, in case of Example 2 where a weight ratio of the wax and the porous powder is 1:0.2, the wax was excessively coated on the inner surface and the outer surface of the porous powder as shown in (e) of FIG. 1, whereas in case of Example 1 where the weight ratio is 1:1, the wax was thinly and uniformly coated on the inner surface and the outer surface of the porous powder as shown in (d) of FIG. 1.

### (2) Confirmation of an Absorption Amount of the Wax-Coated Porous Powder

Based on the wax coating pattern of the porous powder, in order to confirm whether there is a difference in an absorption amount when applied to the skin, after 50 mL of the compositions of Comparative Examples 2 to 4 and Examples 1 and 2 were applied on a membrane (a film that can permeate sebum) and dried, 50 mL of a sebum-mimetic oil was sprayed from a rear surface thereof, and after 30 minutes taking into account the amount absorbed by the artificial skin itself, its pattern was confirmed (FIG. 3).

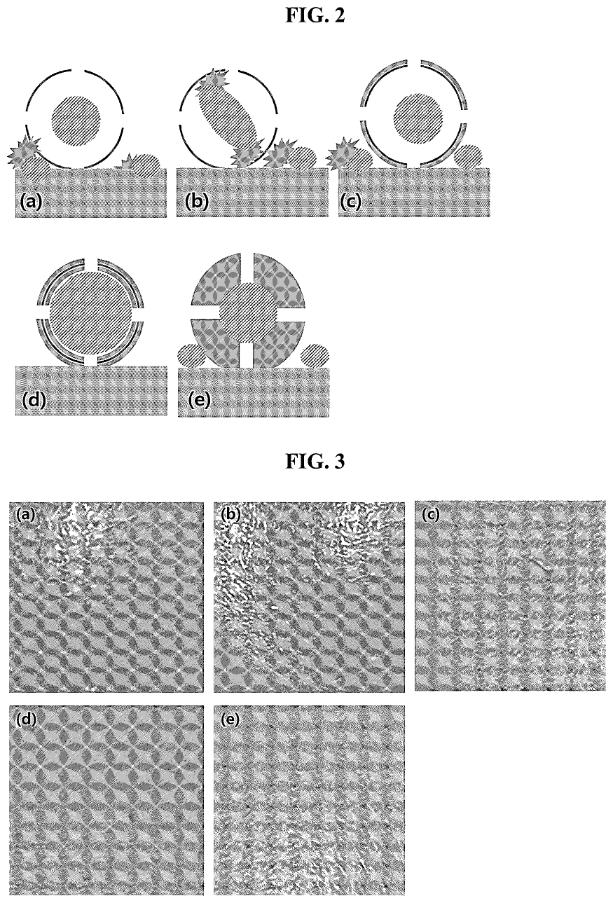

As shown in the schematic diagram of FIG. 2, in case of Comparative Examples 2 and 3, since a surface of the porous powder was not properly coated with the wax, the porous powder having a low affinity for sebum absorbed less sebum, and in case of Comparative Example 4, since the affinity for sebum on the inner surface was low, sebum could not enter the pores efficiently, which results in a small amount of absorption. In case of Example 2, due to excessive coating, there was not enough space inside the porous powder to absorb sebum. On the other hand, in case of Example 1, a thin and uniform wax coating layer was formed, which results in sufficient space inside the porous powder to absorb sebum.

### 4. Sensory Evaluation

The sensory evaluation was conducted with 8 subjects, who applied 50 mL of the contents to the entire face by hand and scored the self-evaluation of glossiness and persistency.

The evaluation score criteria were given the highest score of 5 points for each of the glossiness and the persistency after 8 hours of makeup, with the highest score being given to the more glossiness and the better persistency. The glossiness was evaluated on how much oil was visible, and the persistency was evaluated on the level at which the makeup coverage was maintained. Each was evaluated as 5 points for very strong, 4 points for slightly strong, 3 points for average, 2 points for insufficient, and 1 point for very insufficient. Table 3 shows average scores of the sensory evaluation results for glossiness and persistency for each Example and Comparative Example, and Table 4 shows an individual score for each panel. As a result, the glossiness of Examples 1 and 2 was lower and the persistency was higher, compared to Comparative Examples. In particular, Example 1 showed good results in both glossiness and persistency.

TABLE 3

Sensory Evaluation
(Average Score)

| | Glossiness | Persistency |
|---|---|---|
| Example 1 | 2.5 | 4.7 |
| Example 2 | 3.5 | 3.7 |
| Comparative Example 1 | 4.1 | 2.9 |
| Comparative Example 2 | 4.2 | 2.8 |
| Comparative Example 3 | 4.1 | 3 |
| Comparative Example 4 | 3.6 | 3.3 |

TABLE 4

| | | Panel (Gender/Age) | | | | | | | | |
|---|---|---|---|---|---|---|---|---|---|---|
| | Evaluation | Woman | Woman | Woman | Woman | Woman | Woman | Woman | Man | |
| | Item | 33 | 33 | 30 | 37 | 41 | 32 | 29 | 36 | Average |
| Example 1 | Glossiness | 2 | 3 | 3 | 3 | 2 | 3 | 2 | 2 | 2.5 |
| Example 2 | | 3 | 4 | 3 | 4 | 4 | 4 | 3 | 3 | 3.5 |
| Comparative | | 3 | 4 | 4 | 5 | 5 | 4 | 4 | 4 | 4.1 |
| Example 1 | | | | | | | | | | |
| Comparative | | 5 | 3 | 5 | 5 | 3 | 5 | 4 | 3.5 | 4.2 |
| Example 2 | | | | | | | | | | |
| Comparative | | 4 | 4 | 4 | 5 | 4 | 4 | 4 | 4 | 4.1 |
| Example 3 | | | | | | | | | | |
| Comparative | | 4 | 3 | 4 | 4 | 3 | 4 | 4 | 2.5 | 3.6 |
| Example 4 | | | | | | | | | | |
| Example 1 | Persistency | 5 | 3 | 4 | 4 | 3 | 4 | 3 | 3.5 | 3.7 |
| Example 2 | | 3 | 3 | 3 | 3 | 3 | 4 | 2 | 1.5 | 2.8 |
| Comparative | | 4 | 3 | 2 | 3 | 3 | 3 | 2 | 3 | 2.9 |
| Example 1 | | | | | | | | | | |
| Comparative | | 5 | 4 | 5 | 5 | 5 | 5 | 5 | 3.5 | 4.7 |
| Example 2 | | | | | | | | | | |
| Comparative | | 4 | 3 | 3 | 4 | 3 | 3 | 2 | 2 | 3.0 |
| Example 3 | | | | | | | | | | |
| Comparative | | 3 | 4 | 3 | 4 | 4 | 3 | 3 | 2 | 3.3 |
| Example 4 | | | | | | | | | | |

### 5. Clinical Test

Test subjects were first selected to conduct a clinical test that can evaluate a skin color and uniformity for 24 hours, a coverage persistency, and a skin gloss persistency of the cosmetic composition.

Specifically, 1) women aged 20 to 35, 2) people who voluntarily signed the consent form after hearing a sufficient explanation about the purpose and content of the test, 3) people who can conduct follow-up observation during the test period, and 4) healthy people without acute or chronic physical diseases including skin diseases were selected. Then, among the selected people, the following cases were excluded: 1) Pregnant or lactating women and women of childbearing age who do not agree to the contraceptive method specified in the protocol, 2) people having lesions at the test site, 3) people suffering from infectious skin diseases, 4) people having allergies or hypersensitivity, 5) people having irritation from cosmetics, medicines, or daily light exposure, 6) people who received systemic steroids or phototherapy within 1 month before participating in the test, 7) people who received skin care or procedures within 3 months before participating in the test, 8) people who employed the same or similar cosmetics or medicines on the test site within 3 months prior to the start of the test, 9) people having mental illness, mental retardation, etc., and 10) in addition to the above matters, people who were judged by the principal researcher or the test manager to be difficult to conduct a human test. In addition, even though the test subjects met the above selection criteria and did not fall under the exclusion criteria, they were excluded from the valid data by processing them as a dropout in case of the following: 1) the test subjects voluntarily expressed the intention to discontinue participation in the test, 2) the test subjects developed a skin disease or adverse reaction, 3) the test site excessively exposed to an ultraviolet ray, 4) the test subject having difficulty in evaluating the results due to excessive drinking, smoking, etc., and 5) the test subjects judged to have difficulty in continuing the test or conducting follow-up observation due to their personal circumstances. The number of test subjects was selected and conducted with people of 20 or more based on the Regulations on the Evaluation of Functional Cosmetics of the Ministry of Food and Drug Safety (2015-14, 2015.03.25).

The face site was washed before applying Example 1 and was waited under constant temperature and humidity (20-24° C., 45-55% RH) for 30 minutes, and then was applied with the cosmetic composition of Example 1 once to the entire face site.

The skin color and uniformity for 24 hours, and the coverage persistency on the face site was measured on the front of the face site using VISIA-CR (Canfield Science CO. Ltd, USA) before and immediately after using the product, 12 hours after using the product, and 24 hours after using the product. A I-MAX plus measurement was performed by designating specific sites of the images captured with a general light

### (Standard2 Mode).

The skin gloss persistency for 24 hours was measured on the front of the face site using VISIA-CR (Canfield Science CO. Ltd, USA). The images were taken using a high-resolution camera (Canon 5D) and a 6-position filter wheel at a constant location and environment, and the intensity values were analyzed and evaluated by designating a specific area (face site) using the I-MAX plus program in the captured images (Parallel Polarized mode). The higher the intensity value, the higher an effect of the skin gloss persistency for 24 hours.

### (1) Skin Color-Coverage Persistency, L-Value

Measurement values (L-value) for the skin color-coverage persistency of the test site were confirmed to be increased in a significant level (p<0.05) by 4.179% immediately after use, 3.540% 12 hours after use, and 2.452% 24 hours after use, compared to before use of the cosmetic composition of Example 1, which proves that they had the effect of the skin color-coverage persistency for 24 hours.

| | | | Immediately | 12 hours | 24 hours |
|---|---|---|---|---|---|
| | | Before use | after use | after use | after use |
| Measurement | Average | 76.912 | 80.126 | 79.635 | 78.798 |
| values (L-value) | Standard | 2.974 | 2.101 | 2.133 | 2.436 |
| for skin color- | deviation | | | | |
| coverage | | | | | |
| persistency | | | | | |
| Change rate^{a} | Before use − | 4.179 | | | |
| | Immediately | | | | |
| | after use | | | | |
| | Before use − | 3.540 | | | |
| | 12 hours | | | | |
| | after use | | | | |
| | Before use − | 2.452 | | | |
| | 24 hours | | | | |
| | after use | | | | |
| Significant | Before use − | <0.001* | | | |
| probability | immediately | | | | |
| (p-value) of | after use | | | | |
| comparison | Before use − | <0.001* | | | |
| within group | 12 hours | | | | |
| | after use | | | | |
| | Before use − | <0.001* | | | |
| | 24 hours | | | | |
| | after use | | | | |
| Number of | Before use − | 31 people/100.000% | | | |
| Improved test | 24 hours | | | | |
| subjects/% | after use | | | | |

*p < 005 by repeated measures ANOVA
Change rate^{a} (%): {(Measurement value n hours after using product − Measurement value before using product) / Measurement value before using product × 100}

### (2) Skin Color-Coverage Persistency: A-Value

Measurement values (a-value) for the skin color-coverage persistency of the test site were confirmed to be decreased in a significant level (p<0.05) by 33.406% immediately after use. 30.090% 12 hours after use, and 29.966% 24 hours after use, compared to before use of the cosmetic composition of Example 1, which proves that they had the effect of the skin color-coverage persistency for 24 hours (a-value).

TABLE 6

| | | Before | Immediately | 12 hours | 24 hours |
|---|---|---|---|---|---|
| | | use | after use | after use | after use |
| Measurement | Average | 9.651 | 6.427 | 6.747 | 6.759 |
| values (a-value) | Standard | 2.872 | 1.447 | 1.437 | 1.861 |
| for skin color- | deviation | | | | |
| coverage | | | | | |
| persistency | | | | | |
| Change rate^{a} | Before use − | −33.406 | | | |
| | Immediately | | | | |
| | after use | | | | |
| | Before use − | −30.090 | | | |
| | 12 hours | | | | |
| | after use | | | | |
| | Before use − | −29.966 | | | |
| | 24 hours | | | | |
| | after use | | | | |
| Significant | Before use − | <0.001* | | | |
| probability | immediately | | | | |
| (p-value) of | after use | | | | |
| comparison | Before use − | <0.001* | | | |
| within group | 12 hours | | | | |
| | after use | | | | |
| | Before use − | <0.001* | | | |
| | 24 hours | | | | |
| | after use | | | | |
| Number of | Before use − | 29 people/93.548% | | | |
| Improved test | 24 hours | | | | |
| subjects/% | after use | | | | |

*p < 005 by repeated measures ANOVA
Change rate^{a} (%): {(Measurement value n hours after using product − Measurement value before using product) / Measurement value before using product × 100}

(3) Skin Color-Coverage Persistency, b-Value

Measurement values (b-value) for the skin color-coverage persistency of the test site were confirmed to be decreased in a significant level (p<0.05) by 7.433% immediately after use, 6.558% 12 hours after use, and 8.940% 24 hours after use, compared to before use of the cosmetic composition of Example 1, which proves that they had the effect of the skin color-coverage persistency for 24 hours (b-value).

TABLE 7

| | | Before | Immediately | 12 hours | 24 hours |
|---|---|---|---|---|---|
| | | use | after use | after use | after use |
| Measurement | Average | 17.718 | 16.401 | 16.556 | 16.134 |
| values (b-value) | Standard | 3.032 | 1.249 | 1.645 | 2.163 |
| for skin color- | deviation | | | | |
| coverage | | | | | |
| persistency | | | | | |
| Change rate^{a} | Before use − | −7.433 | | | |
| | Immediately | | | | |
| | after use | | | | |
| | Before use − | −6.558 | | | |
| | 12 hours | | | | |
| | after use | | | | |
| | Before use − | −8.940 | | | |
| | 24 hours | | | | |
| | after use | | | | |
| Significant | Before use − | 0.013* | | | |
| probability | immediately | | | | |
| (p-value) of | after use | | | | |
| comparison | Before use − | 0.042* | | | |
| within group | 12 hours | | | | |
| | after use | | | | |
| | Before use − | 0.003* | | | |
| | 24 hours | | | | |
| | after use | | | | |
| Number of | Before use − | 22 people/70.968% | | | |
| Improved test | 24 hours | | | | |
| subjects/% | after use | | | | |

*: p < 005 by repeated measures ANOVA
Change rate^{a} (%): {(Measurement value n hours after using product − Measurement value before using product) / Measurement value before using product × 100}

### (4) Skin Uniformity-Coverage Persistency, L′Std

Measurement values (L′std) for the uniformity-coverage persistency of the test site were decreased in a significant level (p<0.05) by 8.025% immediately after use, and increased in a significant level (p<0.05) by 20.956% 12 hours after use and 20.790% 24 hours after use, compared to before use of the cosmetic composition of Example 1.

TABLE 8

| | | Before | Immediately | 12 hours | 24 hours |
|---|---|---|---|---|---|
| | | use | after use | after use | after use |
| Measurement | Average | 2.405 | 2.212 | 2.909 | 2.905 |
| values (L′std) | Standard | 0.596 | 0.453 | 0.607 | 0.595 |
| for uniformity- | deviation | | | | |
| coverage | | | | | |
| persistency | | | | | |
| Change rate^{a} | Before use − | −8.025 | | | |
| | Immediately | | | | |
| | after use | | | | |
| | Before use − | 20.956 | | | |
| | 12 hours | | | | |
| | after use | | | | |
| | Before use − | 20.790 | | | |
| | 24 hours | | | | |
| | after use | | | | |
| Significant | Before use − | 0.012*¹ | | | |
| probability | immediately | | | | |
| (p-value) of | after use | | | | |
| comparison | Before use − | <0.001*² | | | |
| within group | 12 hours | | | | |
| | after use | | | | |
| | Before use − | <0.001*² | | | |
| | 24 hours | | | | |
| | after use | | | | |

*p < 0.05 by repeated measures ANOVA
*¹Significant decrease. /
*²Significant increase
Change rate^{a} (%): {(Measurement value n hours after using product − Measurement value before using product) / Measurement value before using product × 100}

(5) Skin Uniformity-Coverage Persistency, a′Std

Measurement values (a′std) for the skin uniformity-coverage persistency of the test site were confirmed to be decreased in a significant level (p<0.05) by 29.479% immediately after use, 13.257% 12 hours after use, and 9.746% 24 hours after use, compared to before use of the cosmetic composition of Example 1, which proves the effect of the skin uniformity-coverage persistency for 24 hours (a′std).

TABLE 9

| | | Before | Immediately | 12 hours | 24 hours |
|---|---|---|---|---|---|
| | | use | after use | after use | after use |
| Measurement | Average | 1.652 | 1.165 | 1.433 | 1.491 |
| values (a′std) | Standard | 0.528 | 0.256 | 0.355 | 0.323 |
| for skin | deviation | | | | |
| uniformity- | | | | | |
| coverage | | | | | |
| persistency | | | | | |
| Change rate^{a} | Before use − | −29.479 | | | |
| | Immediately | | | | |
| | after use | | | | |
| | Before use − | −13.257 | | | |
| | 12 hours | | | | |
| | after use | | | | |
| | Before use − | −9.746 | | | |
| | 24 hours | | | | |
| | after use | | | | |
| Significant | Before use − | <0.001* | | | |
| probability | immediately | | | | |
| (p-value) of | after use | | | | |
| comparison | Before use − | 0.012* | | | |
| within group | 12 hours | | | | |
| | after use | | | | |
| | Before use − | 0.034* | | | |
| | 24 hours | | | | |
| | after use | | | | |
| Number of | Before use − | 19 people/61.290% | | | |
| Improved test | 24 hours | | | | |
| subjects/% | after use | | | | |

*p < 005 by repeated measures ANOVA
Change rate^{a} (%): {(Measurement value n hours after using product − Measurement value before using product) / Measurement value before using product × 100}

(6) Skin Uniformity-Coverage Persistency, b′Std

Measurement values (b′std) for the skin uniformity-coverage persistency of the test site were decreased in a significant level (p<0.05) by 22.725% immediately after use, and increased in a significant level (p<0.05) by 18.775% 24 hours after use, compared to before use of the cosmetic composition of Example 1. This means that the lower the b′std value, the more effective the skin uniformity-persistency effect is.

TABLE 10

| | | Before | Immediately | 12 hours | 24 hours |
|---|---|---|---|---|---|
| | | use | after use | after use | after use |
| Measurement | Average | 2.253 | 1.741 | 2.399 | 2.676 |
| values (b′std) | Standard | 0.390 | 0.310 | 0.627 | 0.630 |
| for skin | deviation | | | | |
| uniformity- | | | | | |
| coverage | | | | | |
| persistency | | | | | |
| Change rate^{a} | Before use − | −22.725 | | | |
| | Immediately | | | | |
| | after use | | | | |
| | Before use − | 6.480 | | | |
| | 12 hours | | | | |
| | after use | | | | |
| | Before use − | 18.775 | | | |
| | 24 hours | | | | |
| | after use | | | | |
| Significant | Before use − | <0.001*¹ | | | |
| probability | immediately | | | | |
| (p-value) of | after use | | | | |
| comparison | Before use − | 0.155 | | | |
| within group | 12 hours | | | | |
| | after use | | | | |
| | Before use − | <0.001*² | | | |
| | 24 hours | | | | |
| | after use | | | | |

*p < 0.05 by repeated measures ANOVA
*¹Significant decrease. /
*²: Significant increase.
Change rate^{a} (%): {(Measurement value n hours after using product. Measurement value before using product) / Measurement value before using product × 100}

### (7) Measurement of Skin Gloss Persistency, Intensity

Measurement values for the skin gloss persistency of the test site were confirmed to be increased in a significant level (p<0.05) by 4.223% immediately after use, 3.182% 12 hours after use, and 2.315% 24 hours after use, compared to before use of the cosmetic composition of Example 1, which proves the effect of the gloss persistency for 24 hours.

TABLE 11

| | | Before | Immediately | 12 hours | 24 hours |
|---|---|---|---|---|---|
| | | use | after use | after use | after use |
| Measurement | Average | 177.805 | 185.314 | 183.463 | 181.922 |
| values | Standard | 8.933 | 7.662 | 8.197 | 8.267 |
| (instensity) | deviation | | | | |
| for skin gloss | | | | | |
| persistency | | | | | |
| Change rate^{a} | Before use − | 4.223 | | | |
| | Immediately | | | | |
| | after use | | | | |
| | Before use − | 3.182 | | | |
| | 12 hours | | | | |
| | after use | | | | |
| | Before use − | 2.315 | | | |
| | 24 hours | | | | |
| | after use | | | | |
| Significant | Before use − | <0.001* | | | |
| probability | immediately | | | | |
| (p-value) of | after use | | | | |
| comparison | Before use − | <0.001* | | | |
| within group | 12 hours | | | | |
| | after use | | | | |
| | Before use − | <0.001* | | | |
| | 24 hours | | | | |
| | after use | | | | |
| Number of | Before use − | 31 people/100.000% | | | |
| Improved test | 24 hours | | | | |
| subjects/% | after use | | | | |

*p < 005 by repeated measures ANOVA
*Change rate^{a} (%): {(Measurement value n hours after using product − Measurement value before using product) / Measurement value before using product × 100}

### Embodiments

Embodiment 1: A pre-dispersed composition for preparing a cosmetic composition for absorbing sebum, the pre-dispersed composition comprising a porous powder, a wax; and a volatile oil, wherein the porous powder and the wax are dispersed in the volatile oil.
Embodiment 2: The pre-dispersed composition according to embodiment 1, wherein a sum of the contents of the porous powder and the wax in the pre-dispersed composition is 2 to 20 wt % based on the total weight of the pre-dispersed composition.
Embodiment 3: The pre-dispersed composition according to embodiment 1 or 2, wherein a weight ratio of the porous powder:the wax:the volatile oil in the pre-dispersed composition is 1:0.25-6:13-23.
Embodiment 4: The pre-dispersed composition according to any one of embodiments 1 to 3, wherein the porous powder is a wax-coated porous powder containing a wax coating layer on its surface, and the surface is an outer surface and an inner surface.
Embodiment 5: The pre-dispersed composition according to any one of embodiments 1 to 4, wherein an average thickness of the wax coating layer is 0.01% to 20% of a diameter of the porous powder.
Embodiment 6: The pre-dispersed composition according to embodiment 4 or 5, wherein the pre-dispersed composition has the volatile oil supported within pores of the wax-coated porous powder.
Embodiment 7: The pre-dispersed composition according to any one of embodiments 1 to 6, characterized in that the wax-coated porous powder has an absorption amount of 0.002 to 0.03 ml/g.
Embodiment 8: The pre-dispersed composition according to any one of embodiments 1 to 7, wherein the porous powder has a surface area of 10 to 500 m²/g.
Embodiment 9: The pre-dispersed composition according to any one of embodiments 1 to 8, wherein the wax is a non-silicone-based wax.
Embodiment 10: The pre-dispersed composition according to any one of embodiments 1 to 9, wherein the wax has a melting point of 40 to 150° C.
Embodiment 11: The pre-dispersed composition according to any one of embodiments 1 to 10, wherein the wax is not separated from a sebum oil in phase after 24 hours from the point of time when the wax is mixed with the sebum oil in a weight ratio of the sebum oil to the wax of 7:3 under a molten state at 90° C. and then reaches a room temperature by natural cooling.
Embodiment 12: The pre-dispersed composition according to any one of embodiments 1 to 11, wherein the volatile oil has a temperature vapor pressure of 0.3 to 90 mmHg at 40° C.
Embodiment 13: The pre-dispersed composition according to any one of embodiments 1 to 12, wherein the cosmetic composition is a makeup cosmetic composition.
Embodiment 14: A cosmetic composition for absorbing sebum, comprising a wax-coated porous powder containing a porous powder and a wax coating layer formed on a surface thereof, wherein the surface of the porous powder is an outer surface and an inner surface.
Embodiment 15: The cosmetic composition according to embodiment 14, wherein a weight ratio of the wax and the porous powder is 1:0.25-6.
Embodiment 16: The cosmetic composition according to embodiment 14 or 15, wherein the wax is a non-silicone-based wax.
Embodiment 17: The cosmetic composition according to any one of embodiments 14 to 16, wherein the wax has a melting point of 40 to 150° C.
Embodiment 18: The cosmetic composition according to any one of embodiments 14 to 17, wherein the wax is not separated from a sebum oil in phase after 24 hours from the point of time when the wax is mixed with the sebum oil in a weight ratio of the sebum oil to the wax of 7:3 under a molten state at 90° C. and then reaches a room temperature by natural cooling.
Embodiment 19: The cosmetic composition according to any one of embodiments 14 to 18, wherein the porous powder has a surface area of 10 to 500 m²/g.
Embodiment 20: The cosmetic composition according to any one of embodiments 14 to 19, wherein the wax-coated porous powder is contained in an amount of more than 0.5 wt % to 5 wt % or less, based on the total weight of the composition.
Embodiment 21: The cosmetic composition according to any one of embodiments 14 to 20, wherein an average thickness of the wax coating layer is 0.01% to 20% of a diameter of the porous powder.
Embodiment 22: The cosmetic composition according to any one of embodiments 14 to 21, characterized in that the wax-coated porous powder has an absorption amount of 0.002 to 0.03 ml/g.
Embodiment 23: The cosmetic composition according to any one of embodiments 14 to 22, further comprising a volatile oil.
Embodiment 24: The cosmetic composition according to embodiment 23, wherein the wax-coated porous powder has the volatile oil supported within pores thereof.
Embodiment 25: The cosmetic composition according to any one of embodiment 23 or 24, wherein the volatile oil has a temperature vapor pressure of 0.3 to 90 mmHg at 40° C.
Embodiment 26: The cosmetic composition according to any one of embodiments 14 to 25, further comprising a non-volatile solvent.
Embodiment 27: The cosmetic composition according to any one of embodiments 14 to 26, further comprising at least one of a dispersant and a non-absorbent powder.
Embodiment 28: The cosmetic composition according to any one of embodiments 14 to 27, wherein the cosmetic composition is a makeup cosmetic composition.
Embodiment 29: The cosmetic composition according to embodiment 28, characterized in that the makeup cosmetic composition is a formulation selected from the group consisting of a makeup primer, a foundation, a makeup base, and a tinted moisturizer.
Embodiment 30: The cosmetic composition according to any one of embodiments 14 to 29, wherein the cosmetic composition is prepared from the pre-dispersed composition of claims 1 to 12.
Embodiment 31: A method for preparing the cosmetic composition according to any one of embodiments 14 to 30, the method comprising the steps of: (a) preparing a pre-dispersed composition; and (b) dispersing a result product of step (a) in a non-volatile solvent.
Embodiment 32: The method for preparing the cosmetic composition according to embodiment 31, wherein step (a) includes (i) adding a porous powder and a wax to a volatile oil; and (ii) dispersing the porous powder and the wax using a milling machine or a mixer.
Embodiment 33: The method for preparing the cosmetic composition according to embodiment 31 or 32, wherein step (b) includes adding 2 to 20 wt % of the pre-dispersed composition produced by step (a) based on the total weight of the cosmetic composition.
Embodiment 34: The method for preparing the cosmetic composition according to any one of embodiments 31 to 33, wherein step (a) is performed at 40 to 150° C.

1-25. (canceled)

26. A method for preparing a cosmetic composition for absorbing sebum comprising the steps of:
(a) preparing a pre-dispersed composition comprising a porous powder; a wax; and a volatile oil, wherein the porous powder and the wax are dispersed in the volatile oil and
(b) dispersing a result product of step (a) in a non-volatile solvent.

27. The method according to claim 26,
wherein a sum of the contents of the porous powder and the wax in the pre-dispersed composition is 2 to 20 wt % based on the total weight of the pre-dispersed composition.

28. The method according to claim 26,
wherein a weight ratio of the porous powder:the wax:the volatile oil in the pre-dispersed composition is 1:0.25-6:13-23.

29. The method according to claim 26,
wherein the porous powder is a wax-coated porous powder comprising a wax coating layer on its surface, and
the surface is an outer surface and an inner surface.

30. The method according to claim 29,
wherein an average thickness of the wax coating layer is 0.01% to 20% of a diameter of the porous powder, or
the pre-dispersed composition has the volatile oil supported within pores of the wax-coated porous powder, or
characterized in that the wax-coated porous powder has an absorption amount of 0.002 to 0.03 ml/g based on the weight of the porous powder.

31. The method according to claim 26,
wherein the porous powder has a surface area of 10 to 500 m²/g.

32. The method according to claim 26,
wherein the wax is a non-silicone-based wax, or
the wax has a melting point of 40 to 150° C., or
the wax is not separated from a sebum oil in phase after 24 hours from the point of time when the wax is mixed with the sebum oil in a weight ratio of the sebum oil to the wax of 7:3 under a molten state at 90° C. and then reaches a room temperature by natural cooling.

33. The method according to claim 26,
wherein the volatile oil has a temperature vapor pressure of 0.3 to 90 mmHg at 40° C.

34. The method according to claim 26,
wherein the cosmetic composition is a makeup cosmetic composition.

35. The method according to claim 26,
wherein step (a) comprises:
(i) adding a porous powder and a wax to a volatile oil; and
(ii) dispersing the porous powder and the wax using a milling machine or a mixer, or wherein step (a) is performed at 40 to 150° C.

36. The method according to claim 26,
wherein step (b) comprises adding 2 to 20 wt % of the pre-dispersed composition produced by step (a), based on the total weight of the cosmetic composition.

37. A method for absorbing sebum in a skin of a subject in need thereof, which comprises administering an effective amount of a cosmetic composition comprising a wax-coated porous powder to the subject in need thereof,
wherein the wax-coated porous powder comprises a porous powder and a wax coating layer formed on a surface thereof,
wherein the surface of the porous powder is an outer surface and an inner surface.

38. The method according to claim 37,
wherein a weight ratio of the porous powder and the wax is 1:0.25-6.

39. The method according to claim 37,
wherein the wax-coated porous powder is comprised in an amount of more than 0.5 wt % to 5 wt % or less, based on the total weight of the cosmetic composition.

40. The method according to claim 37, wherein the cosmetic composition further comprises a non-volatile solvent or at least one of a dispersant and a non-absorbent powder.

41. The method according to claim 37,
wherein the cosmetic composition is a makeup cosmetic composition.

42. The method according to claim 41,
characterized in that the cosmetic composition is a formulation selected from the group consisting of a makeup primer, a foundation, a makeup base, and a tinted moisturizer.

43. The method according to claim 37,
wherein the cosmetic composition is prepared by a method comprising the steps of:
(a) preparing a pre-dispersed composition comprising a porous powder; a wax; and a volatile oil, wherein the porous powder and the wax are dispersed in the volatile oil and
(b) dispersing a result product of step (a) in a non-volatile solvent.
